(19) United States
(12) Patent Application Publication
(10) Pub. No.: US 2009/0137880 A1
(43) Pub. Date: May 28, 2009

(54) Determining the Risk of Breast Cancer for a Woman

(75) Inventors: John Boyages, New South Wales (AU); Nathan Coombs, Wiltshire (GB); Richard Taylor, Queensland (AU)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: SYDNEY WEST AREA HEALTH SERVICE, Westmead (AU)

(21) Appl. No.: 11/910,318

(22) PCT Filed: Aug. 29, 2005
(86) PCT No.: PCT/AU05/01296

§ 371 Date: Jul. 24, 2008

(30) Foreign Application Priority Data
Apr. 1, 2005 (AU) 2005901619

(51) Int. Cl. A61B 5/00

(52) U.S. Cl. 600/300

(57) ABSTRACT

A method (100) of determining cumulative absolute risk of breast cancer for a woman is disclosed. The method (100) assists women and doctors to make informed decisions about the additional risk of breast cancer from HRT use. The disclosed method and the implementation of the methods as software such as an application program executing within a computer system may also be used for many other diseases.

### FIELD OF THE INVENTION

The present invention relates generally to cancer and, in particular, to a method and apparatus for determining the risk of breast cancer for a woman, and to a computer program product including a computer readable medium having recorded thereon a computer program for determining the risk of breast cancer for a woman.

### BACKGROUND

Absolute breast cancer risk for women is a combination of relative risks: inherited, physiological and acquired. Whilst a woman's baseline inherited risk will not change, factors such as lifestyle, fertility, diet and medication may modify subsequent risk. Hormone replacement therapy (HRT) has been identified as a risk factor for breast cancer development. Recent large studies and randomized controlled trials have demonstrated that both type and duration of HRT use affect a women's risk of developing breast cancer. A randomized controlled study entitled “Writing Group for the Women's Health Initiative Investigators. Risks and benefits of estrogen plus progestin in healthy postmenopausal women: principal results From the Women's Health Initiative randomized controlled trial” by Rossouw et al, JAMA. 2002; 288: 321-33 (“the WHI study”) was stopped prematurely because the risk of breast cancer exceeded the benefits of combination HRT for fracture prevention. The WHI study estimated a breast cancer relative risk (RR) of 1.26 for a woman who has had HRT for a period of five years. Evidence from a study entitled “Million Women Study Collaborators. Breast cancer and hormone-replacement therapy in the Million Women Study”, by Beral V, Lancet. 2003; 362: 419-27 (“the UK Million Women Study”) suggests that current HRT use increases a woman's relative risk RR of developing breast cancer by 1.66.

Several studies have stated that combined Oestrogen-Progestogen HRT therapy poses a higher breast cancer risk than Oestrogen-only therapy and a more recent study investigating the effects of conjugated estrogen in postmenopausal women with hysterectomy suggests a reduction in breast cancer risk.

The reported higher risk of breast cancer due to HRT has caused concern. There has been a reduction in the prevalence of HRT use in post-menopausal women in the light of recent publicity. In Australia, women aged 55-59 years are the highest users of HRT and between 1991 and 2001, the proportion of women over the age of 50 years on HRT doubled to 28%. This prevalence of HRT use is similar to that in large UK studies.

Doctors and women need to know the absolute risk of breast cancer in the remaining years of a woman's life. Lifetime risk data, from birth to average life expectancy, are freely available. However, specific breast cancer risk data for an individual is not available and cannot be easily determined. Such specific risk data is required for an individual in a clinical context where cumulative absolute risk declines because years of remaining life diminish, even though the age-specific risk increases.

Thus, a need clearly exists for a more efficient method of determining the risk of breast cancer for a woman.

### SUMMARY

It is an object of the present invention to substantially overcome, or at least ameliorate, one or more disadvantages of existing arrangements.

Disclosed is a method which allows doctors and women to determine cumulative absolute risk of breast cancer in relation to HRT use.

According to one aspect of the present invention there is provided a method of determining cumulative absolute breast cancer risk for a patient, said method comprising the steps of:

determining underlying population breast cancer incidence associated with said patient;

determining baseline age specific breast cancer incidence for said patient based on said underlying population breast cancer incidence; and

determining the cumulative absolute breast cancer risk for said patient based on the baseline age specific breast cancer incidence determined for said patient, wherein the underlying population breast cancer incidence associated with said patient is adjusted for hormone replacement therapy (HRT) use.

According to another aspect of the present invention there is provided an apparatus for determining cumulative absolute breast cancer risk for a patient, said apparatus comprising:

means for determining underlying population breast cancer incidence associated with said patient;

means for determining baseline age specific breast cancer incidence for said patient based on said underlying population breast cancer incidence; and

means for determining the cumulative absolute breast cancer risk for said patient based on the baseline age specific breast cancer incidence determined for said patient.

According to still another aspect of the present invention there is provided a computer program product including a computer readable medium having recorded thereon a computer program for determining cumulative absolute breast cancer risk for a patient, said program comprising:

code for determining underlying population breast cancer incidence associated with said patient;

code for determining baseline age specific breast cancer incidence for said patient based on said underlying population breast cancer incidence; and

code for determining the cumulative absolute breast cancer risk for said patient based on the baseline age specific breast cancer incidence determined for said patient.

According to still another aspect of the present invention there is provided a graphical user interface displayed on a computer display for use in determining cumulative absolute breast cancer risk for a patient, said interface comprising:

means for entering details of said patient including one or more of age details, hormone replacement therapy details and family history;

means for displaying underlying population breast cancer incidence associated with said patient based on the entered patient details;

means for displaying baseline age specific breast cancer incidence for said patient, said age specific breast cancer risk being determined based on said underlying population breast cancer incidence; and

means for displaying the cumulative absolute breast cancer risk for said patient, said cumulative absolute breast cancer risk being determined based on the baseline age specific breast cancer incidence determined for said patient

According to still another aspect of the present invention there is provided a method of determining cumulative absolute breast cancer risk for a patient, said method comprising the steps of:

determining underlying population breast cancer incidence associated with said patient;

determining baseline age specific breast cancer incidence for said patient based on said underlying population breast cancer incidence; and

determining the cumulative absolute breast cancer risk for said patient based on the baseline age specific breast cancer incidence determined for said patient.

According to still another aspect of the present invention there is provided a method of determining cumulative absolute risk of a particular disease for a patient based on a particular risk factor, said method comprising the steps of:

determining underlying population incidence associated with said patient for the particular disease;

determining baseline age specific incidence of the particular disease for the patient based on the underlying population incidence; and

determining the cumulative absolute risk for the patient based on the baseline age specific incidence determined for the person, wherein the underlying population incidence associated with the patient is adjusted for the risk factor.

According to still another aspect of the present invention there is provided an apparatus for determining cumulative absolute risk of a particular disease for a patient based on a particular risk factor, said apparatus comprising:

means for determining underlying population incidence associated with said patient for the particular disease;

means for determining baseline age specific incidence of the particular disease for the patient based on the underlying population incidence; and

means for determining the cumulative absolute risk for the patient based on the baseline age specific incidence determined for the person, wherein the underlying population incidence associated with the patient is adjusted for the risk factor.

According to still another aspect of the present invention there is provided a computer program product including a computer readable medium having recorded thereon a computer program for determining cumulative absolute risk of a particular disease for a patient based on a particular risk factor, said program comprising:

code for determining underlying population incidence associated with said patient for the particular disease;

code for determining baseline age specific incidence of the particular disease for the patient based on the underlying population incidence; and

code for determining the cumulative absolute risk for the patient based on the baseline age specific incidence determined for the person, wherein the underlying population incidence associated with the patient is adjusted for the risk factor.

According to still another aspect of the present invention there is provided a graphical user interface displayed on a computer display for determining cumulative absolute risk of a particular disease for a patient based on a particular risk factor, said interface comprising:

means for entering details of said patient including one or more of age details, risk factor details and family history;

means for displaying underlying population disease incidence associated with said patient based on the entered patient details;

means for displaying baseline age specific disease incidence for said patient, said age specific disease incidence being determined based on said underlying population disease incidence; and

means for displaying the cumulative absolute disease risk for said patient, said cumulative absolute disease risk being determined based on the baseline age specific disease incidence determined for said patient

Other aspects of the invention are also disclosed.

### BRIEF DESCRIPTION OF THE DRAWINGS

Some aspects of the prior art and one or more embodiments of the present invention will now be described with reference to the drawings and appendices, in which:

FIG. 1 shows a method of determining a cumulative absolute risk of breast cancer for a woman;

FIG. 2 is a schematic block diagram of a general purpose computer upon which the method of FIG. 1 may be practiced;

FIG. 3 shows a user interface for use in one implementation of the method of FIG. 1;

FIG. 4 shows a method of determining a cumulative absolute risk of a disease for a person based on a risk factor.

### DETAILED DESCRIPTION INCLUDING BEST MODE

Where reference is made in any one or more of the accompanying drawings to steps and/or features, which have the same reference numerals, those steps and/or features have for the purposes of this description the same function(s) or operation(s), unless the contrary intention appears.

It is to be noted that the discussions contained in the “Background” section and that above relating to prior art arrangements relate to discussions of documents or devices which form public knowledge through their respective publication and/or use. Such should not be interpreted as a representation by the present inventor(s) or patent applicant that such documents or devices in any way form part of the common general knowledge in the art.

Reported population incidence of breast cancer is based on a combination of risks including baseline risk, risk attributable to genetics and family history, risk associated with medication (e.g., HRT therapy) and risk associated with lifestyle decisions. A variable representing the effect of HRT therapy or family history on the risk of contracting breast cancer may be referred to as an attributable fraction (AF). The attributable fraction AF may be removed from reported population incidence values before a baseline risk is determined for a woman, as will be described in detail below.

HRT and other risk factors normally associated with breast cancer may influence absolute risk less in an elderly population because the number of years remaining at risk is less than in a younger population. Age seventy nine (79) years is used as an upper limit in the described methods because age seventy nine (79) years approximates to the average life expectancy of women in Australia and because after this age mortality is very high from multiple competing causes.

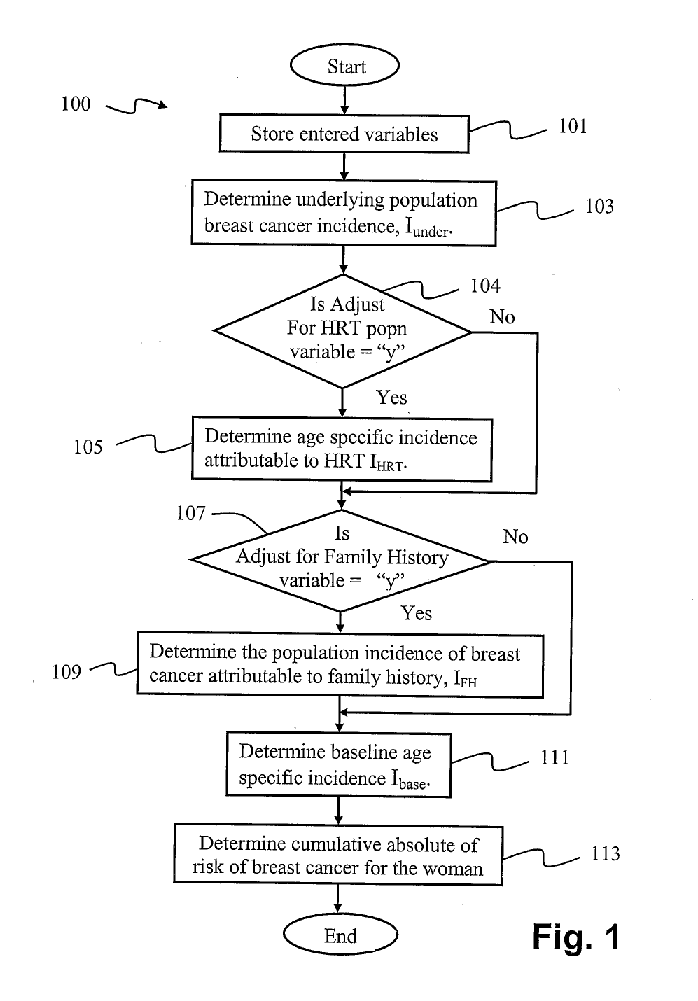

A method 100 (see FIG. 1) of determining cumulative absolute risk of breast cancer for a woman is described below with reference to FIGS. 1 to 3. The method 100 assists women and doctors to make informed decisions about the additional risk of breast cancer from HRT use. This risk may be balanced against the potential quality of life benefits of taking HRT to reduce the symptoms of menopause. The method 100 allows women and doctors to make informed decisions regarding treatment options during menopause. The method 100 also provides a woman with information for her and other members of her family about the probability of developing breast cancer.

The principles of the method 100 have general applicability to determining breast cancer risk for all women. However, for ease of explanation, the steps of the method 100 are described with reference to the Australian population. It is not intended that the present invention be limited to the described method 100. For example, the invention may have application to other people groups (e.g., other Western countries like the United Kingdom, Europe and the United States of America). The methods described herein and the implementation of those methods as software, such as an application program executing within a computer system may also be used for many other diseases, as will be described below.

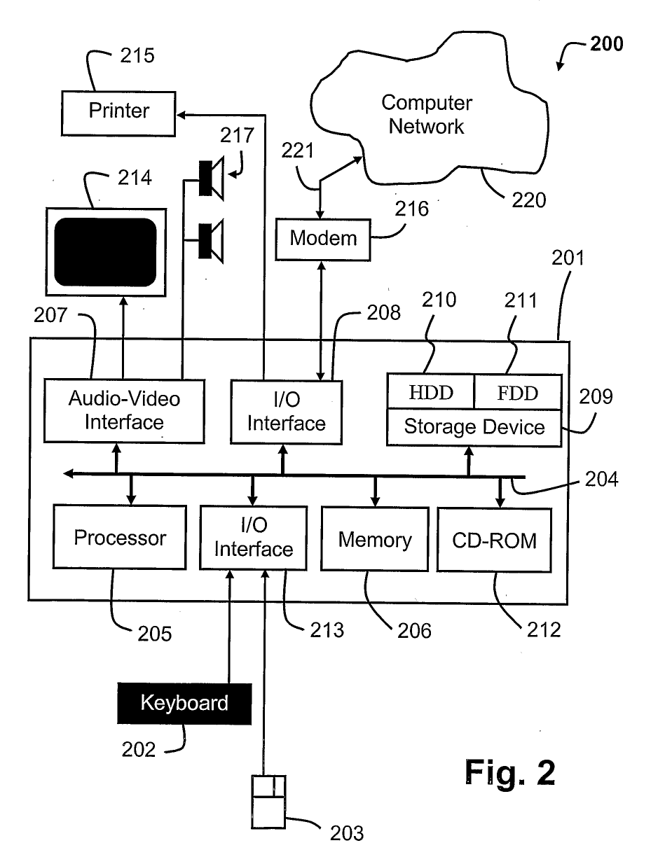

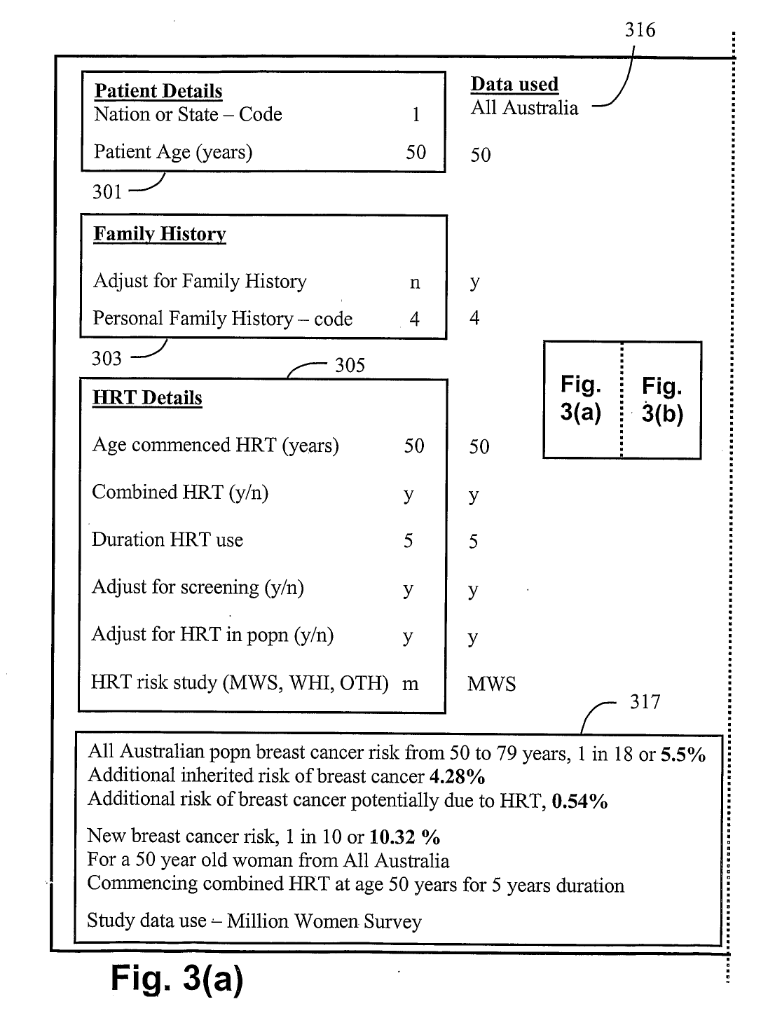

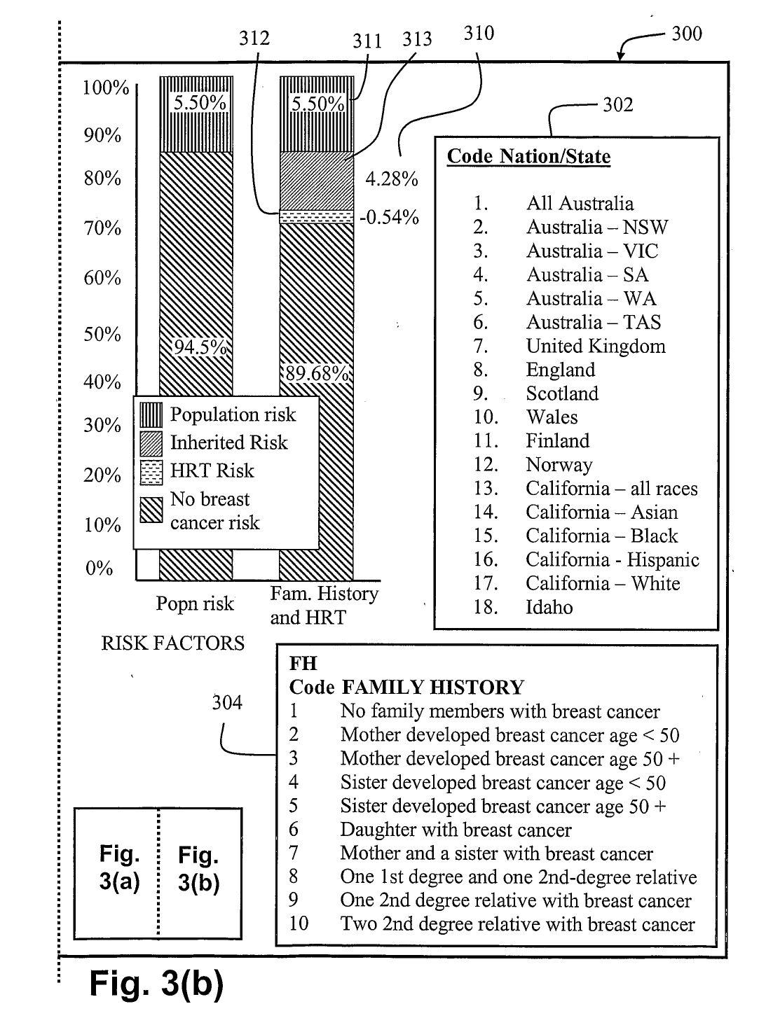

The method 100 may be practiced using a general-purpose computer system 200, such as that shown in FIG. 2 wherein the processes of FIG. 1 may be implemented as software, such as an application program executing within the computer system 200. In particular, the steps of the method 100 may be effected by instructions in the software that are carried out by the computer system 200. The instructions may be formed as one or more code modules, each for performing one or more particular tasks. The software may also be divided into two separate parts, in which a first part performs the described method 100 and a second part manages a user interface between the first part and the user. FIG. 3 shows a user interface 300, which may be used in one implementation of the method 100.

The software may be stored in a computer readable medium, including the storage devices described below, for example. The software may be loaded into the computer from the computer readable medium, and then executed by the computer. A computer readable medium having such software or computer program recorded on it is a computer program product. The use of the computer program product in the computer preferably effects an advantageous apparatus for implementing the described methods.

The computer system 200 is formed by a computer module 201, input devices such as a keyboard 202 and mouse 203, output devices including a printer 215, a display device 214 and loudspeakers 217. A Modulator-Demodulator (Modem) transceiver device 216 is used by the computer module 201 for communicating to and from a communications network 220, for example connectable via a telephone line 221 or other functional medium. The modem 216 can be used to obtain access to the Internet, and other network systems, such as a Local Area Network (LAN) or a Wide Area Network (WAN), and may be incorporated into the computer module 201 in some implementations.

The computer module 201 typically includes at least one processor unit 205, and a memory unit 206, for example formed from semiconductor random access memory (RAM) and read only memory (ROM). The module 201 also includes an number of input/output (I/O) interfaces including an audio-video interface 207 that couples to the video display 214 and loudspeakers 217, an I/O interface 213 for the keyboard 202 and mouse 203 and optionally a joystick (not illustrated), and an interface 208 for the modem 216 and printer 215. In some implementations, the modem 2116 may be incorporated within the computer module 201, for example within the interface 208. A storage device 209 is provided and typically includes a hard disk drive 210 and a floppy disk drive 211. A magnetic tape drive (not illustrated) may also be used. A CD-ROM drive 212 is typically provided as a non-volatile source of data. The components 205 to 213 of the computer module 201, typically communicate via an interconnected bus 204 and in a manner which results in a conventional mode of operation of the computer system 200 known to those in the relevant art. Examples of computers on which the described arrangements can be practised include IBM-PC's and compatibles, Sun Sparcstations or alike computer systems evolved therefrom. The described arrangements may also be practised using portable computers such as palm sized calculators, laptops, mobile phones and the like.

Typically, the application program is resident on the hard disk drive 210 and read and controlled in its execution by the processor 205. Intermediate storage of the program and any data fetched from the network 220 may be accomplished using the semiconductor memory 206, possibly in concert with the hard disk drive 210. In some instances, the application program may be supplied to the user encoded on a CD-ROM or floppy disk and read via the corresponding drive 212 or 211, or alternatively may be read by the user from the network 220 via the modem device 216. Still further, the software may also be loaded into the computer system 200 from other computer readable media. The term “computer readable medium” as used herein refers to any storage or transmission medium that participates in providing instructions and/or data to the computer system 200 for execution and/or processing. Examples of storage media include floppy disks, magnetic tape, CD-ROM, a hard disk drive, a ROM or integrated circuit, a magneto-optical disk, or a computer readable card such as a PCMCIA card and the like, whether or not such devices are internal or external of the computer module 201. Examples of transmission media include radio or infra-red transmission channels as well as a network connection to another computer or networked device, and the Internet or Intranets including e-mail transmissions and information recorded on Websites and the like.

The method 100 may alternatively be implemented in dedicated hardware such as one or more integrated circuits performing the functions or sub functions of the described arrangements. Such dedicated hardware may include graphic processors, digital signal processors, or one or more microprocessors and associated memories.

The method 100 of determining the cumulative absolute risk of breast cancer for a woman will now be described in detail with reference to FIG. 1. The method 100 will be described with reference to FIG. 3, which shows the user interface 300 used in one implementation of the method 300.

The user interface 300 may be used by a doctor to enter a number of variables for use by the method 100 in determining the cumulative absolute risk of breast cancer for a woman. Labels corresponding to each of the variables are listed below as follows:

(i) Nation or State code;

(ii) Patient age (years);

(iii) Adjust for Family History;

(iv) Personal Family History-code;

(v) Age Commenced HRT (years);

(vi) Combined HRT (y/n);

(vii) Duration HRT use;

(viii) Adjust for Screening (y/n);

(ix) Adjust for HRT in popn (y/n); and

(x) HRT risk study (MWS, WHI, OTH).

Values for each of the variables represented by the above labels may be entered by the doctor using the keyboard 202 adjacent to a respective label on the user interface 300. Upon being entered, the values for each of the variables may be stored in memory 206 by the processor 205 for use during execution of the method 100.

The user interface 300 includes an area 301 entitled “Patient Details” for entering the details of the woman for whom the method 100 is to be executed. In the Patient Details area 301, a doctor may enter the age of the woman (i.e., adjacent to the label “Patient age (years)”) and a code (i.e., adjacent to the label “Nation or State-code”) representing the nation or state of birth for the woman. As seen in FIG. 3, a list of Nation or State codes 302 is provided on the user interface 300. The list of codes 302 includes eighteen nations or states including Australia (code 1), New South Wales (code 2) and California—all races (code 13). The list of codes 302 represents nations and states for which age specific breast cancer incidence data is available. This breast cancer incidence data may be stored on the hard disk drive 210. Breast cancer incidence data may be obtained from various sources including the Cancer Council NSW website (www.nswcc.org.au). For example, attached as Appendix A is a table showing the recorded incidence of breast cancer (Iₚₒₚ) in NSW in 2001.

The user interface 100 also includes an area 303 entitled “Family History” for entering family history information for the woman. In the area 303, the doctor may enter in yes (y) or no (n) (i.e., adjacent to the label “Adjust for Family History”) to indicate whether the method 100 should take into account family history for the woman when determining the cumulative absolute risk of breast cancer for the woman. The doctor may also enter in a personal history code (i.e., adjacent to the label “Personal Family History-code”) for the woman, representing the family history of the woman. As seen in FIG. 3, a list of Family History codes 304 is provided on the user interface 300. The list of codes 304 includes ten codes indicating the personal family history of the woman as follows:

(i) FH Code 1: No family members with breast cancer;

(ii) FH Code 2: Mother who developed breast cancer aged <50 years;

(iii) FH Code 3: Mother who developed breast cancer aged 50+ years;

(iv) FH Code 4: Sister who developed breast cancer aged <50 years;

(v) FH Code 5: Sister who developed breast cancer aged 50+ years;

(vi) FH Code 6: Daughter with breast cancer;

(vii) FH Code 7: Mother and sister with breast cancer;

(viii) FH Code 8: One 1^{st} degree and one 2^{nd} degree relative;

(ix) FH Code 9: One second degree relative with breast cancer; and

(x) FH Code 10: Two second degree relatives with breast cancer.

The user interface 300 also includes an area 305 entitled “HRT Details” for entering in details of any HRT therapy that the woman is undergoing. In the area 305, the doctor may enter in (i.e., adjacent to the label “Age Commenced HRT (years)”) the age in years at which the woman commenced HRT therapy. The doctor may also enter in yes (y) or no (n) (i.e., adjacent to the label “Combined HRT (y/n)”) to indicate whether the woman was on combined HRT therapy or not, respectively, and the duration of HRT use in years (i.e., adjacent to the label “Duration HRT use”).

Reported population breast cancer incidence data may be determined by the use of widespread breast screening. However, the detection of small, impalpable breast tumors by mammography increases the reported breast cancer incidence for women of screening age incidence by “borrowing” cancer incidence from the older generation. The area 305 may also be used to enter in yes (y) or no (n) (i.e., adjacent to the label “Adjust for Screening (y/n)”) to indicate whether the method 100 should take into account breast cancer screening (i.e., screening effect) in regard to reported population age-specific breast cancer incidence being used in the method 100. The screening effect and age-specific breast cancer incidence will be described in more detail below.

The area 305 may also be used to enter in yes (y) or no (n) (i.e., adjacent to the label “Adjust for HRT in popn (y/n)”) to indicate whether the impact of HRT therapy on reported breast cancer incidence in a population should be taken into account in determining the cumulative absolute risk of breast cancer for the woman. Finally, the area 305 may also be used to enter in (i.e., adjacent to the label “HRT risk study (MWS, WHI, OTH)” the HRT risk study data that should be used by the method 100. The doctor may select the UK Million Women Study (MWS) or the WHI study. Results of the UK Million Women Study and the WHI study may be stored on the hard disk drive 210 prior to executing the method 100. Alternatively, the doctor may decide to use other information (OTH) in the method 100. In this instance, the doctor stores this other information on the hard disk drive 210 prior to executing the method 100.

The method 100 begins at step 101, where upon being entered by the doctor the processor 205 stores values in memory 206 for one or more of the above variables. The values entered by the doctor and the values used by the method 100 are display in a horizontal column 316 under the heading “Date used”. If the doctor does not enter a value for one or more of the above variables, then the processor 205 may select default values for the variables which were not entered. These default values may be stored on the hard disk drive 210 prior to the execution of the method 100. For example, the default value for the Adjust for Family History variable may be “n” and the default value for the Personal Family History-code variable may be “1” indicating that the woman has no family members with breast cancer. Any suitable default values may be used.

At the next step 103, the processor 205 determines underlying population breast cancer incidence (I_{under}) for the woman. The underlying population breast cancer incidence I_{under} is dependent on reported population age-specific breast cancer incidence (Iₚₒₚ). The underlying population breast cancer incidence I_{under} is also dependent on estimates of the effect of breast screening on breast cancer incidence, which is represented by a variable referred to as “screening effect”. The underlying population breast cancer incidence I_{under} may be determined in accordance with Formula (1) below:

I_{under}=Iₚₒₚ×(1−screening effect) (1)

The age-specific breast cancer incidence Iₚₒₚ is retrieved by the processor 205, from the breast cancer incidence data stored on the hard disk drive 210. The value selected by the processor 205 is dependent on the Patient Age (years) variable (i.e., representing the age of the woman) and the Nation or State-code (i.e., the code representing the nation or state of birth of the woman), stored in memory 206. The age of the woman and the age-specific breast cancer incidence data used by the method 100 is also indicated in an area 317 of the user interface.

In one example, the doctor may have selected to adjust the age-specific breast cancer incidence value Iₚₒₚ based on the screening effect, by entering in yes (“y”) or no (“n”) adjacent to the label Adjust for Screening in the area 303. If the value stored in memory 206 for the Adjust for Screening variable is “y”, then the processor 205 also determines a value for screening effect based on the value of the Nation or State-code variable stored in memory 205.

The value for screening effect may also be retrieved by the processor 205 from screening effect by age data stored on the hard disk drive 210, Appendix B is a table showing age-specific screening effect values for an Australian population. Appendix B was sourced from a publication entitled “Estimating risk of breast cancer from population incidence affected by widespread mammographic screening”, J Med Screening 2001; 8: 73-6, by R Taylor and J. Boyages.

Therefore, if the Nation or State-code variable stored in memory 205 is “1” and the value of the Patient Age (years) variable is “50” then the processor 205 will select a value of 0.1755 (i.e., 17.55%) for the screening effect variable.

The table of Appendix A also shows the underlying incidence of breast cancer I_{under} adjusted for screening in NSW in 2001. The annual incidence of breast cancer in Australia is about 11,000 new cases per year, of which over 4,000 are diagnosed in NSW by the Australian Institute of Health and Welfare. The State of NSW is one of six federated States and Territories of Australia with a population of approximately 6.6 million, 34% of the population of Australia. In a 2001 National Census, 4.4 million people in NSW (66.7%) stated that they were Australian-born. Of those born overseas, the main countries of birth were the United Kingdom (4.4%) and New Zealand (1.7%). The data presented in Appendix A would be applicable to other Western countries where breast cancer is prevalent.

The value for the underlying population breast cancer incidence I_{under} determined by the processor 205 at step 103 may be stored in memory 206. At the next step 104, if the value of the Adjust for HRT in popn (y/n) variable is equal to “y”, then the method 100 proceeds to step 105. Otherwise, the method 100 proceeds to step 107. At step 105, the processor 205 determines a value representing age-specific breast cancer incidence attributable to HRT (I_{HRT}) using the value of I_{under} determined in step 103. The I_{HRT} represents the underlying population breast cancer incidence I_{under} with the effect of HRT on the breast cancer incidence in a population removed. The effect of HRT on the breast cancer incidence in a population may be referred to as the Attributable Factor (AF). The AF may be determined using direct and indirect methods. The AF may be determined in accordance with the indirect method using Formula (2) below:

$$AF = \frac{p ( RR - 1 )}{p ( RR - 1 ) + 1} \qquad ( 2 )$$

where RR represents the relative risk of a woman contracting breast cancer based on the family history of the woman and p represents the prevalence of different types of HRT use in the population.

The prevalence p may be determined in accordance with Formula (3) as follows:

$$p = HRT prevalence \times \begin{matrix}proportion of use by \\ HRT medication type\end{matrix} \qquad ( 3 )$$

The prevalence of HRT use (i.e., HRT prevalence) and the proportion of use by HRT medication type may be estimated by national women's health surveys and is available for a number of populations. For example, the prevalence of HRT use in Australia was estimated from a quinquennial Australian Health Survey the (“AHS”) carried out in 2001. This national survey involved 50,000 people and contained an additional Women's Supplementary Health Form administered to 9750 women over the age of 18 years that questioned, inter alia, their HRT status and duration of HRT use. Result data from the AHS was extrapolated to provide estimates of proportion and duration of HRT use for the entire Australian population by five-year age group. This allows an estimation of age-specific HRT prevalence. For each age group, the duration of HRT use was sub-divided according to the number of years of treatment (i.e., <1 year, 1-5 years, 5-10 years, 10 or more years). The AHS also provided weighted estimates of numbers of HRT users for the Australian population. Where some sub-divisions contained small numbers, age groupings were collapsed; <45 years, five-yearly age groups to 65 years, and 65+ years. Appendix C is a table showing HRT use in Australia based on age group (i.e., 20-44 years, 45-49 years, 50-54 years, 55-59 years, 60-64 years and 65+) and years of treatment. Appendix C also shows the numbers of HRT users for each of the age groups mentioned directly above.

As seen from Appendix C, in 2001, 11.7% of adult women aged 18-80 years were current users of HRT and use of HRT was highest in the postmenopausal group with a peak prevalence of 38% in 55-59 year-olds. Over one quarter of Australian women between 50 and 65 years were current users of HRT and in the 55-65 age group, over two-thirds of HRT users had received HRT for at least five years.

For women aged 50 years or less, most took HRT for less than five years. Prolonged HRT, for more than 10 years duration, was most common in women aged over 55 years, and in current users aged 60 years or older, over one half of these women had been taking HRT for more than 10 years.

Information about the relative risk (RR) for breast cancer with HRT use may be obtained from the WHI study, the UK Million Women Study and a meta-analysis of 51 HRT studies. Relative risk RR values derived from the UK Million Women Study and the WHI studies according to HRT type and duration of use are shown in Appendices D and E, respectively. The relative risk RR values of Appendices D and E may be stored in the hard disk drive 210 and accessed by the processor 205. A summary and comparison of the principle findings of each of the WHI, the UK Million Women and the meta-analysis of 51 HRT studies is shown in Appendix F.

Use of the information from the UK Million Women Study and WHI study shown in Appendices D and E provide specific values for relative risk RR according to HRT type and duration of use. The processor 205 determines which study to retrieve values for relative risk RR from based on the value of the HRT risk study variable stored in memory 206. For example, if the HRT risk study variable is set to “m” and the Combined HRT (y/n) variable is set to “y”, then the processor 205 will determine values for relative risk RR from the table of Appendix D entitled “Million Women Survey: Combination HRT (estrogen and progesterone)”. The study used by the method 100 is indicated in the area 317 of the user interface.

The attributable factor AF may be estimated using the direct method in accordance with Formula (4) below:

$$AF = \frac{{I}_{under} - {I}_{never}}{{I}_{under}} \qquad ( 4 )$$

where I_{under} represents the reported breast cancer incidence within a population, adjusted for screening, as described above, and Iₙₑᵥₑᵣ represents baseline breast cancer incidence in non-users of HRT. A value representing age-specific underlying incidence of breast cancer in a population who have never been exposed to HRT may also be determined by combining the direct and indirect AF methodologies. The baseline breast cancer incidence in non-users of HRT (Iₙₑᵥₑᵣ) may be determined in accordance with Formula (5) below

$${I}_{never} = {I}_{under} [ 1 - ( \frac{p ( RR - 1 )}{P ( RR - 1 ) + 1} ) ] \qquad ( 5 )$$

The age-specific incidence attributable to HRT (I_{HRT}) is determined from I_{under} and Iₙₑᵥₑᵣ in accordance with Formula (6) below:

I_{HRT}=I_{under}−Iₙₑᵥₑᵣ (6)

The method continues at step 107, where if the processor 205 determines that the Adjust for Family History variable is set to “y” then the method 100 proceeds to step 109. Otherwise, the method 100 proceeds to step 111. At step 109, the processor 205 determines the population incidence of breast cancer attributable to family history (I_{FH}). The population incidence of breast cancer attributable to family history I_{FH} may be determined in accordance with Formula (7), below:

I_{FH}=I_{under}−I_{noFH} (7)

where I_{noFH} represents incidence of breast cancer with no family history, and may be determined in accordance with Formula (8) below:

$${I}_{noFH} = {I}_{under} [ 1 - ( \frac{p ( RR - 1 )}{p ( RR - 1 ) + 1} ) ] \qquad ( 8 )$$

where RR represents the relative risk of a woman contracting breast cancer based on the family history of the woman and p represents the prevalence of different types of HRT use in the population. The values for p and RR attributable to family history were sourced from the publication entitled “Family History and the risk of breast cancer. A systematic review and meta-analysis”. by Pharoah P, Day N, Duffy S, Easton D, Ponder B, Int J Cancer 1997; 71: 800-9”, and may be stored in the hard disk drive 210. The values for RR selected by the processor 205 at step 109 will be dependent on the value of the Personal Family History-code variable. Appendix G is a table showing relative risk values for each of the Personal Family History codes. The table of Appendix G may be stored in the hard disk drive 210 and be accessed by the processor 205 when required.

At the next step 111, the processor 205 determines a value for baseline age-specific breast cancer incidence (I_{base}) from the age-specific reported incidence I_{under} and incidence attributable to HRT (I_{HRT}) and family history I_{FH} in accordance with Formula (8) below:

I_{base}=I_{under}−(I_{HRT}+I_{FH}) (8)

The method 100 continues at the next step 113, where the processor 205 determines the cumulative absolute risk of breast cancer for the woman in accordance with Formula (9), below:

Cumulative absolute risk=1−e^{(-Cumulative rate)} (9)

where cumulative rate is the sum from a specific age to 79 years of the yearly age-specific baseline breast cancer incidence I_{base} in the population. Different populations may have different risks of developing breast cancer in the subsequent years of life due to varying reported breast cancer incidence rates between populations and different effects of HRT and family history on the incidence. The method 100 concludes following step 113.

Attached as Appendix H is a table showing absolute cumulative risk of breast cancer incidence in non-HRT users, determined in accordance with the method 100, for an Australian woman.

As seen in the table of Appendix H, the absolute cumulative risk of breast cancer incidence in non-HRT users (from 40 to 79 years) is approximately 7.2% (1 in 14), reducing to 6.1% (1 in 16) at 50 years and to approximately 4.4% (1 in 23) at 60 years. Breast cancer relative risk RR values may be applied to age-specific breast cancer incidence rates for different HRT duration and type scenarios and to determine additional breast cancer risk of HRT. As seen in the table of Appendix H, use of Oestrogen-only HRT or short-term (˜5 years) use of combined HRT commencing at the age of 50 years will hardly affect the cumulative absolute breast cancer risk determined to the age of 79 (no HRT: 6.1%, Oestrogen-only: 6.3%, Oestrogen+Progesterone: 6.7%). Prolonged (˜10 years) use of Oestrogen+Progesterone HRT will increase the cumulative risk to 7.7%.

As also seen in the table of Appendix H, a 55-year-old woman has a cumulative absolute breast cancer risk to age 79 years of 5.3% (1 in 19). Five years of Oestrogen-only HRT, will impart an additional risk of breast cancer of 0.2%. Increasing the duration of HRT increases the additional breast cancer risk. Ten and fifteen years of Oestrogen-only HRT commenced at age 55 years will increase this woman's breast cancer risk further by 0.5% and 0.9% respectively.

The additional breast cancer risk is more considerable with combination HRT, especially if taken for more than five years. As seen in the table of Appendix H, five years of Oestrogen+Progesterone HRT therapy, commencing at the age of 55 years, will generate an extra 0.6% breast cancer risk and 10 years a further 1.8% risk. Once HRT is terminated the relative risk quickly returns to 1.0 and the cumulative absolute risk of breast cancer returns to that of an age-matched non-user.

Values for the age-specific baseline breast cancer incidence I_{base} may be used to determine the incidence of breast cancer for years between decade and mid-decade ages. For example, breast cancer incidence at 52 years (I₅₂) may be determined from the breast cancer incidence at 50 years (I₅₀) and breast cancer incidence at 55 years (I₅₅) in accordance with Formula (9) below:

$${I}_{52} = {I}_{50} + [ ( \frac{52 - 50}{5} ) \times ( {I}_{55} - {I}_{50} ) ] \qquad ( 9 )$$

Once the baseline breast cancer incidence is determined relative risk RR values for HRT of different formulations and duration of use may be applied. The relative risk values applied are taken from published research that can stratify the breast cancer relative risk by duration of use and formulation. Relative risk values derived from the UK Million Women Study and the WHI studies are shown in Appendices D and E, respectively, as described above. As also described above, the relative risk values are selected by the processor 205 from the appropriate UK Million Women Study and the WHI study depending on the value of the HRT risk study variable stored in memory 206. The selected relative risk values may be applied to the baseline age-specific breast cancer incidence values I_{base} to determine new breast cancer values incidence if a hypothetical cohort of women should all take HRT of a certain type for a specific duration, commencing in a certain year. From the new calculated incidence, the cumulative absolute breast cancer risk may be determined.

As an example, the baseline age-specific breast cancer incidence I_{base} and new breast cancer incidence I_{new} for a 50 year old Australian women who wishes to commence combination HRT (i.e., estrogen and progesterone) at age 50 for a total of 5 years duration, are shown in Table 1 below. The cumulative absolute breast cancer risk to age 79 years is also shown in Table 1.

TABLE 1

| | | Base cumulative rate | | | | New cumulative rate | | Additional |
|---|---|---|---|---|---|---|---|---|
| Age | I_{base} | and risk | | HRT RR | I_{new} | and risk | | Risk |
| 50 | 143.1 | 0.057 | 5.5% | 1 | 143.1 | 0.062 | 6.0% | 0.5% |
| 51 | 149.8 | 0.055 | 5.4% | 1.45 | 217.2 | 0.061 | 5.9% | 0.5% |
| 52 | 156.4 | 0.054 | 5.2% | 1.595 | 249.5 | 0.059 | 5.7% | 0.5% |
| 53 | 156.4 | 0.052 | 5.1% | 1.74 | 272.2 | 0.056 | 5.5% | 0.4% |
| 54 | 156.4 | 0.051 | 4.9% | 1.826 | 285.7 | 0.053 | 5.2% | 0.3% |
| 55 | 156.5 | 0.049 | 4.8% | 1.912 | 299.1 | 0.051 | 4.9% | 0.2% |
| 56 | 156.5 | 0.047 | 4.6% | 1.14 | 178.4 | 0.048 | 4.7% | 0.0% |
| 57 | 156.5 | 0.046 | 4.5% | 1 | 156.5 | 0.046 | 4.5% | 0.0% |
| 58 | 160.2 | 0.044 | 4.3% | 1 | 160.2 | 0.044 | 4.3% | 0.0% |
| 59 | 163.8 | 0.043 | 4.2% | 1 | 163.8 | 0.043 | 4.2% | 0.0% |
| 60 | 167.5 | 0.041 | 4.0% | 1 | 167.5 | 0.041 | 4.0% | 0.0% |
| 65 | 184.5 | 0.032 | 3.2% | 1 | 184.5 | 0.032 | 3.2% | 0.0% |
| 70 | 216.0 | 0.023 | 2.2% | 1 | 216.0 | 0.023 | 2.2% | 0.0% |
| 75 | 224.1 | 0.011 | 1.1% | 1 | 224.1 | 0.011 | 1.1% | 0.0% |
| 79 | 239.5 | 0.002 | 0.2% | 1 | 239.5 | 0.002 | 0.2% | 0.0% |

A comparison of the baseline risk I_{base} and new risk I_{new} may be made and the additional risk may be determined in accordance with Formula (10):

Additional Risk=I_{ew}−I_{base} (10)

The relative risks RR values due to family history may be determined from published research and applied to the baseline age-specific breast cancer incidence I_{base}. The relative risk RR values used in the method 100 are listed in Appendix G for different combinations of family history, as described above, and the additional risk attributable to a family history may be determined. For example, a 50 year old Australian woman is concerned about her risk of breast cancer. She has a sister who developed breast cancer before the age of 50 years.

The baseline age-specific breast cancer incidence I_{base} and additional cumulative absolute breast cancer risk values may be summed to provide a total risk of breast cancer to 79 years for a woman of a specific age. This total risk of breast cancer value may be printed or demonstrated graphically as a bar graph 310 on the user interface 300 to compare how the total risk is comprised of a baseline component 311, HRT component 312 and family history component 313. For example, as displayed on the user interface 300, a 50 year old Australian, commencing 5 years of combination HRT, with a sister who developed breast cancer before the age of 50 years has a cumulative absolute breast cancer risk of 10.2%. This cumulative absolute breast risk is determined from the baseline risk (5.5%) displayed by the portion 311, family history risk (4.2%) displayed by the portion 313 and risk potentially attributable to HRT (0.54%) displayed by the portion 312. The cumulative absolute breast risk determined from the baseline risk (5.5%), family history risk (4.2%) and risk potentially attributable to HRT (0.54%), is also displayed in the area 317 of the user interface 300.

The method 100 produces information that is useful for the general public and in a clinical setting and quotes absolute cumulative risks from a specific age to age 79 years with and without HRT. Absolute risks for an individual are more meaningful and intuitively understandable than relative risks that require an appreciation of a woman's existing risk. This provides more relevant information than annual or five- or 10-year probabilities or risk from birth as women present for advice at various ages and their main interest is in their risk of developing breast cancer in the remaining years of their life. The absolute risk will decrease as the period over which the risk will be experienced continues to decrease with advancing years. The use of cumulative risks is similar to known actuarial life table methods and is useful for quantifying what may happen to a hypothetical cohort if it passed through the age-specific rates used in the determinations.

Most women are aware of recent HRT publicity. Although this may heighten anxiety of breast cancer risk, the absolute risk of developing breast cancer for an individual may not be as high as assumed and HRT may be terminated prematurely, leading to a possible reduction in quality of life. Conversely, other women may under-estimate their additional cancer risk and continue to take HRT, falsely believing that they have a low probability of breast cancer.

As described above, for the method 100, the relative risk values RR were derived from the UK Million Women Study. Although the results of the UK Million Women Study have been criticized as overstating the relative risk RR of HRT due to detection bias, similar relative risks were estimated from recent randomized controlled trials, a major meta-analysis of international studies and older cohort studies. The relative risk RR is not influenced by local breast cancer incidence and thus, should apply to an Australian population, for example. Use of information from this very large cohort provides data with smaller standard errors and allowed sub-group analysis according to HRT formulation and duration of use.

Printed copies of breast cancer risk and additional risk attributable to HRT at decade and mid-decade ages may be generated for use. Such printed copies may be used in general practitioner (GP) settings. Further, as described above, the data listed in each of the tables of Appendices A to H, are stored in the hard disk drive 210. However, in one implementation, the tables of Appendices A to H may be stored on a remote server. In this instance, the tables of Appendices A to H may be accessed by the processor 205, via the communications network 220.

The methods described above and the implementation of those methods as software, such as an application program executing within the computer system 200, may also be used for many other diseases. For example, the software implemented methods may be used to determine the cumulative absolute risk of lung cancer for a smoker. In this instance, a similar user interface to the user interface 300 described above may be used by a doctor to enter a number of variables for use by a method of determining the cumulative absolute risk of lung cancer for a smoker. Further, lung cancer incidence data may be stored on the hard disk drive 210 in a similar manner to the breast cancer incidence data described above. Again, lung cancer incidence data may be obtained from various sources including the Cancer Council NSW website (www.nswcc.org.au). Smoking risk study data may also be accessed from various studies on the risks of smoking for lung cancer. The user interface used for such methods may include an area, similar to the area 305, which may also be used to enter in which smoking risk study data that should be used by the methods. The doctor may select an appropriate study. Alternatively, the doctor may decide to use other information in the methods. In this instance, the doctor stores this other information on the hard disk drive 210 prior to executing the methods.

Still further, the relative risk of a person contracting lung cancer based on the family history of the person may also be determined and used in determining the cumulative absolute risk of lung cancer for a smoker.

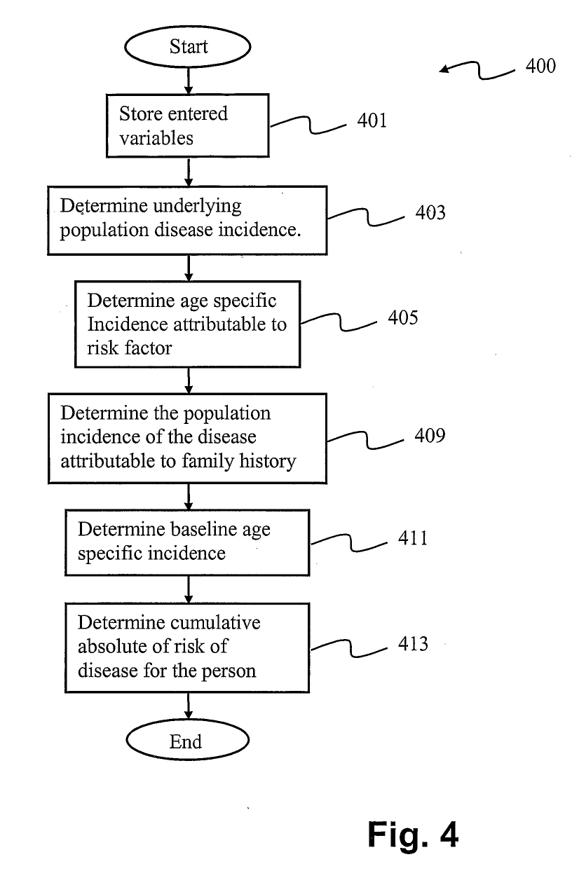

FIG. 4 shows a method 400 of determining a cumulative absolute risk of a disease (e.g., lung cancer) for a person based on a risk factor (e.g., smoking). The method 400 may be used to assist people and doctors to make informed decisions about the additional risk of cancer or other diseases based on risk factors, such as smoking. This risk may be balanced against the potential quality of life benefits of the risk factors. The method 400 allows people and doctors to make informed decisions regarding treatment options. The method 400 also provides a person with information for them and other members of their family about the probability of developing disease.

The method 400 may be practiced using a general-purpose computer system 200, such as that shown in FIG. 2 wherein the processes of FIG. 4 may be implemented as software, such as an application program executing within the computer system 200. Again, the method 400 may be implemented using a user interface (not shown) similar to the user interface 300 of FIG. 3. Such a user interface may be used by a doctor to enter a number of variables for use by the method 400 in determining the cumulative absolute risk of disease for the person. For example, where the disease is smoking related the following labels corresponding to each of a number of variables as listed below may be used:

(i) Nation or State code;

(ii) Patient age (years);

(iii) Adjust for Family History;

(iv) Personal Family History-code;

(v) Age Commenced smoking (years);

(vi) Duration smoking use; and

(vii) Smoking risk study.

Values for each of the variables represented by the above labels may be entered by the doctor using the keyboard 202 adjacent to a respective label on the user interface 300. Upon being entered, the values for each of the variables may be stored in memory 206 by the processor 205 for use during execution of the method 400.

The doctor may also enter in a personal history code for the person, representing the family history of the person. A list of Family History codes may be provided on the associated user interface used for the method 400.

The user interface used for the method 400 may also include an area for entering in details of any risk factor associated with that person. The doctor may also enter in the age in years at which the person became associated with the risk factor (e.g., year began smoking, number of cigarettes per day, exposure to cigarette smoke and years of exposure).

The method 400 begins at step 401, where upon being entered by the doctor the processor 205 stores values in memory 206 for one or more of the above variables. The values entered by the doctor and the values used by the method 400 may be displayed on the appropriate user interface.

At the next step 403, the processor 205 determines underlying population disease incidence for the person in a similar manner to the determination of the underlying population breast cancer incidence described above.

At step 405, the processor 205 determines a value representing age-specific disease incidence attributable to the risk factor using the underlying population disease incidence for the person determined in step 403. The value representing age-specific disease incidence attributable to the risk factor may be determined using an attributable factor similar to the Attributable Factor (AF) described above for breast cancer and may include a relative risk attribute representing the relative risk of the contracting the disease based on the family history of the person. Information about the relative risk (RR) for the disease with the risk factor may be obtained from relevant studies.

At step 409, the processor 205 determines the population incidence of the disease attributable to family history. The population incidence of the disease attributable to family history may be determined in similar to the determination of the population incidence of breast cancer attributable to family history I_{FH} described above.

At the next step 411, the processor 205 determines a value for baseline age-specific disease incidence from the age-specific reported incidence and incidence attributable to the risk factor and family history, in similar manner to the determination of the baseline age-specific breast cancer incidence (I_{base}) described above.

The method 100 continues at the next step 413, where the processor 205 determines the cumulative absolute risk of the disease for the person in a similar manner to the determination of the cumulative absolute risk of breast cancer for a woman, described above.

The aforementioned preferred method(s) comprise a particular control flow. There are many other variants of the preferred method(s) which use different control flows without departing the spirit or scope of the invention. Furthermore one or more of the steps of the preferred method(s) may be performed in parallel rather than sequentially.

### INDUSTRIAL APPLICABILITY

It is apparent from the above that the arrangements described are applicable to the medical industries.

The foregoing describes only some embodiments of the present invention, and modifications and/or changes can be made thereto without departing from the scope and spirit of the invention, the embodiments being illustrative and not restrictive.

In the context of this specification, the word “comprising” means “including principally but not necessarily solely” or “having” or “including”, and not “consisting only of”. Variations of the word “comprising”, such as “comprise” and “comprises” have correspondingly varied meanings.

APPENDIX A
Recorded and “underlying” incidence of breast cancer NSW women in 2001

| | | | Population | | | | |
|---|---|---|---|---|---|---|---|
| | Recorded | Underlying | cumulative risk to age | | | Cumulative risk in group | |
| Age | rate* | rate† | 79 years | | HRT non- | HRT non-users | |
| (years) | (Iₚₒₚ) | (I_{under}) | % | 1 in | users rate‡ | % | 1 in |
| 25-29 | 8.3 | 8.2 | 8.8 | 11 | 8.2 | 7.7 | 13 |
| 30-34 | 28.3 | 25.4 | 8.7 | 11 | 25.4 | 7.7 | 13 |
| 35-39 | 60.9 | 60.5 | 8.6 | 12 | 60.5 | 7.5 | 13 |
| 40-44 | 114.7 | 103.0 | 8.4 | 12 | 102.4 | 7.3 | 14 |
| 45-49 | 175.9 | 152.3 | 7.9 | 13 | 142.7 | 6.8 | 15 |
| 50-54 | 251.0 | 206.7 | 7.2 | 14 | 174.4 | 6.1 | 16 |
| 55-59 | 297.5 | 253.4 | 6.2 | 16 | 177.9 | 5.3 | 19 |
| 60-64 | 305.9 | 253.1 | 5.1 | 19 | 199.6 | 4.4 | 23 |
| 65-69 | 294.7 | 259.1 | 3.9 | 25 | 229.1 | 3.5 | 29 |
| 70-74 | 326.5 | 289.5 | 2.7 | 37 | 256.0 | 2.4 | 42 |
| 75-79 | 280.8 | 255.6 | 1.3 | 78 | 226.0 | 1.1 | 88 |

NSW: New South Wales
Incidence: cases/100 000 population
*Source: NSW breast cancer incidence for 2001 supplied by the NSW Central Cancer Registry.
†The “underlying” incidence of breast cancer in 2001 was estimated from an age, period, cohort model based on 1972-1996 data, using the average of the stable period effect 1972-1989 6.21.
‡Breast Cancer incidence in non-users of hormone replacement therapy (I never) estimated using the Attibutable Fraction method. In the age groups 25-39 years, where 1-IRT use is negligible, the underlying breast cancer incidence is used.

APPENDIX B
Magnitude of the age-specific screening
effect in an Australian Population

| Age | |
|---|---|
| (years) | Screening effect |
| Under 5 | |
| 5-9 | |
| 10-14 | |
| 15-19 | |
| 20-24 | |
| 25-29 | |
| 30-34 | |
| 35-39 | |
| 40-44 | 10.59% |
| 45-49 | 19.16% |
| 50-54 | 17.55% |
| 55-59 | 22.16% |
| 60-64 | 24.94% |
| 65-69 | 20.03% |
| 70-74 | 12.19% |
| 75-79 | 17.49% |
| 80-84 | |
| 85+ | |

APPENDIX C
HRT use in Australia by duration and age group.

| Age | | | | | | | | | Total HRT | | Non |
|---|---|---|---|---|---|---|---|---|---|---|---|
| group | <1 year | | 1-<5 years | | 5-<10 years | | 10+years | | users | | users |
| (years) | ‘000 | % | ‘000 | % | ‘000 | % | ‘000 | % | ‘000 | % | ‘000 |
| 20-44 | 13.68 | 0.4 | 20.35 | 0.6 | 4.54 | 0.1 | 6.69 | 0.2 | 45.26 | 1.4 | 3200.2 |
| 45.49 | 26.95 | 4.4 | 38.88 | 6.3 | 13.94 | 2.3 | 7.86 | 1.3 | 87.63 | 14.2 | 531.23 |
| 50.54 | 23.71 | 4.1 | 84.63 | 14.5 | 36.40 | 6.2 | 26.40 | 4.5 | 171.14 | 29.3 | 412.69 |
| 55.59 | 8.32 | 1.9 | 40.24 | 9.2 | 55.30 | 12.6 | 61.13 | 14.0 | 164.99 | 37.7 | 272.85 |
| 60.64 | 4.30 | 1.2 | 18.95 | 5.1 | 29.68 | 8.0 | 61.10 | 16.5 | 114.03 | 30.9 | 255.18 |
| 65+ | 3.25 | 0.4 | 20.85 | 2.5 | 28.46 | 3.5 | 80.10 | 9.7 | 132.66 | 16.1 | 691.57 |

Source: Australian Health Survey - 2001.
Duration and age-specific HRT prevalence estimated as a percentage of users within each group

APPENDIX D

| Year | No | | | | | | | | | | | | | | | |
|---|---|---|---|---|---|---|---|---|---|---|---|---|---|---|---|---|
| of use | HRT | 1 | 2 | 3 | 4 | 5 | 6 | 7 | 8 | 9 | 10 | 11 | 12 | 13 | 14 | 15 |

Million Women Survey: Estrogen only HRT

| | | | | | | | | | | | | | | | | |
|---|---|---|---|---|---|---|---|---|---|---|---|---|---|---|---|---|
| 0 | 1 | 1 | 1 | 1 | 1 | 1 | 1 | 1 | 1 | 1 | 1 | 1 | 1 | 1 | 1 | 1 |
| 1 | 1 | 0.81 | 0.81 | 0.81 | 0.81 | 0.81 | 0.81 | 0.81 | 0.81 | 0.81 | 0.81 | 0.81 | 0.81 | 0.81 | 0.81 | 0.81 |
| 2 | 1 | 1.14 | 1.03 | 1.03 | 1.03 | 1.03 | 1.03 | 1.03 | 1.03 | 1.03 | 1.03 | 1.03 | 1.03 | 1.03 | 1.03 | 1.03 |
| 3 | 1 | 1 | 1.14 | 1.25 | 1.25 | 1.25 | 1.25 | 1.25 | 1.25 | 1.25 | 1.25 | 1.25 | 1.25 | 1.25 | 1.25 | 1.25 |
| 4 | 1 | 1 | 1 | 1.14 | 1.26 | 1.26 | 1.26 | 1.26 | 1.26 | 1.26 | 1.26 | 1.26 | 1.26 | 1.26 | 1.26 | 1.264 |
| 5 | 1 | 1 | 1 | 1 | 1.14 | 1.28 | 1.28 | 1.28 | 1.28 | 1.28 | 1.28 | 1.28 | 1.28 | 1.28 | 1.28 | 1.278 |
| 6 | 1 | 1 | 1 | 1 | 1 | 1.14 | 1.29 | 1.29 | 1.29 | 1.29 | 1.29 | 1.29 | 1.29 | 1.29 | 1.29 | 1.292 |
| 7 | 1 | 1 | 1 | 1 | 1 | 1 | 1.14 | 1.31 | 1.31 | 1.31 | 1.31 | 1.31 | 1.31 | 1.31 | 1.31 | 1.306 |
| 8 | 1 | 1 | 1 | 1 | 1 | 1 | 1 | 1.14 | 1.32 | 1.32 | 1.32 | 1.32 | 1.32 | 1.32 | 1.32 | 1.32 |
| 9 | 1 | 1 | 1 | 1 | 1 | 1 | 1 | 1 | 1.14 | 1.35 | 1.35 | 1.35 | 1.35 | 1.35 | 1.35 | 1.353 |
| 10 | 1 | 1 | 1 | 1 | 1 | 1 | 1 | 1 | 1 | 1.14 | 1.39 | 1.39 | 1.39 | 1.39 | 1.39 | 1.385 |
| 11 | 1 | 1 | 1 | 1 | 1 | 1 | 1 | 1 | 1 | 1 | 1.14 | 1.42 | 1.42 | 1.42 | 1.42 | 1.418 |
| 12 | 1 | 1 | 1 | 1 | 1 | 1 | 1 | 1 | 1 | 1 | 1 | 1.14 | 1.45 | 1.45 | 1.45 | 1.45 |
| 13 | 1 | 1 | 1 | 1 | 1 | 1 | 1 | 1 | 1 | 1 | 1 | 1 | 1.14 | 1.45 | 1.45 | 1.45 |
| 14 | 1 | 1 | 1 | 1 | 1 | 1 | 1 | 1 | 1 | 1 | 1 | 1 | 1 | 1.14 | 1.45 | 1.45 |
| 15 | 1 | 1 | 1 | 1 | 1 | 1 | 1 | 1 | 1 | 1 | 1 | 1 | 1 | 1 | 1.14 | 1.45 |
| 16 | 1 | 1 | 1 | 1 | 1 | 1 | 1 | 1 | 1 | 1 | 1 | 1 | 1 | 1 | 1 | 1.14 |
| 17 | 1 | 1 | 1 | 1 | 1 | 1 | 1 | 1 | 1 | 1 | 1 | 1 | 1 | 1 | 1 | 1 |

Million Women Survey: Combination HRT (estrogen and progesterone)

| | | | | | | | | | | | | | | | | |
|---|---|---|---|---|---|---|---|---|---|---|---|---|---|---|---|---|
| 0 | 1 | 1 | 1 | 1 | 1 | 1 | 1 | 1 | 1 | 1 | 1 | 1 | 1 | 1 | 1 | 1 |
| 1 | 1 | 1.45 | 1.45 | 1.45 | 1.45 | 1.45 | 1.45 | 1.45 | 1.45 | 1.45 | 1.45 | 1.45 | 1.45 | 1.45 | 1.45 | 1.45 |
| 2 | 1 | 1.14 | 1.60 | 1.60 | 1.60 | 1.60 | 1.60 | 1.60 | 1.60 | 1.60 | 1.60 | 1.60 | 1.60 | 1.60 | 1.60 | 1.60 |
| 3 | 1 | 1 | 1.14 | 1.74 | 1.74 | 1.74 | 1.74 | 1.74 | 1.74 | 1.74 | 1.74 | 1.74 | 1.74 | 1.74 | 1.74 | 1.74 |
| 4 | 1 | 1 | 1 | 1.14 | 1.83 | 1.83 | 1.83 | 1.83 | 1.83 | 1.83 | 1.83 | 1.83 | 1.83 | 1.83 | 1.83 | 1.826 |
| 5 | 1 | 1 | 1 | 1 | 1.14 | 1.91 | 1.91 | 1.91 | 1.91 | 1.91 | 1.91 | 1.91 | 1.91 | 1.91 | 1.91 | 1.912 |
| 6 | 1 | 1 | 1 | 1 | 1 | 1.14 | 2.00 | 2.00 | 2.00 | 2.00 | 2.00 | 2.00 | 2.00 | 2.00 | 2.00 | 1.998 |
| 7 | 1 | 1 | 1 | 1 | 1 | 1 | 1.14 | 2.08 | 2.08 | 2.08 | 2.08 | 2.08 | 2.08 | 2.08 | 2.08 | 2.084 |
| 8 | 1 | 1 | 1 | 1 | 1 | 1 | 1 | 1.14 | 2.17 | 2.17 | 2.17 | 2.17 | 2.17 | 2.17 | 2.17 | 2.17 |
| 9 | 1 | 1 | 1 | 1 | 1 | 1 | 1 | 1 | 1.14 | 2.21 | 2.21 | 2.21 | 2.21 | 2.21 | 2.21 | 2.205 |
| 10 | 1 | 1 | 1 | 1 | 1 | 1 | 1 | 1 | 1 | 1.14 | 2.24 | 2.24 | 2.24 | 2.24 | 2.24 | 2.24 |
| 11 | 1 | 1 | 1 | 1 | 1 | 1 | 1 | 1 | 1 | 1 | 1.14 | 2.28 | 2.28 | 2.28 | 2.28 | 2.275 |
| 12 | 1 | 1 | 1 | 1 | 1 | 1 | 1 | 1 | 1 | 1 | 1 | 1.14 | 2.31 | 2.31 | 2.31 | 2.31 |
| 13 | 1 | 1 | 1 | 1 | 1 | 1 | 1 | 1 | 1 | 1 | 1 | 1 | 1.14 | 2.31 | 2.31 | 2.31 |
| 14 | 1 | 1 | 1 | 1 | 1 | 1 | 1 | 1 | 1 | 1 | 1 | 1 | 1 | 1.14 | 2.31 | 2.31 |
| 15 | 1 | 1 | 1 | 1 | 1 | 1 | 1 | 1 | 1 | 1 | 1 | 1 | 1 | 1 | 1.14 | 2.31 |
| 16 | 1 | 1 | 1 | 1 | 1 | 1 | 1 | 1 | 1 | 1 | 1 | 1 | 1 | 1 | 1 | 1.14 |
| 17 | 1 | 1 | 1 | 1 | 1 | 1 | 1 | 1 | 1 | 1 | 1 | 1 | 1 | 1 | 1 | 1 |

Values in bold are published relative risks. Other values are estimated risks derived from published values

APPENDIX E

| Year | No | | | | | | | | | | | | | | | |
|---|---|---|---|---|---|---|---|---|---|---|---|---|---|---|---|---|
| of use | HRT | 1 | 2 | 3 | 4 | 5 | 6 | 7 | 8 | 9 | 10 | 11 | 12 | 13 | 14 | 15 |

Women's Health Initiative Study: Estrogen only HRT

| | | | | | | | | | | | | | | | | |
|---|---|---|---|---|---|---|---|---|---|---|---|---|---|---|---|---|
| 0 | 1 | 1 | 1 | 1 | 1 | 1 | 1 | 1 | 1 | 1 | 1 | 1 | 1 | 1 | 1 | 1 |
| 1 | 1 | 1.00 | 1.00 | 1.00 | 1.00 | 1.00 | 1.00 | 1.00 | 1.00 | 1.00 | 1.00 | 1.00 | 1.00 | 1.00 | 1.00 | 1.00 |
| 2 | 1 | 1.14 | 1.00 | 1.00 | 1.00 | 1.00 | 1.00 | 1.00 | 1.00 | 1.00 | 1.00 | 1.00 | 1.00 | 1.00 | 1.00 | 1.00 |
| 3 | 1 | 1 | 1.14 | 1.00 | 1.00 | 1.00 | 1.00 | 1.00 | 1.00 | 1.00 | 1.00 | 1.00 | 1.00 | 1.00 | 1.00 | 1.00 |
| 4 | 1 | 1 | 1 | 1.14 | 1.00 | 1.00 | 1.00 | 1.00 | 1.00 | 1.00 | 1.00 | 1.00 | 1.00 | 1.00 | 1.00 | 1 |
| 5 | 1 | 1 | 1 | 1 | 1.14 | 1.00 | 1.00 | 1.00 | 1.00 | 1.00 | 1.00 | 1.00 | 1.00 | 1.00 | 1.00 | 1 |
| 6 | 1 | 1 | 1 | 1 | 1 | 1.14 | 1.00 | 1.00 | 1.00 | 1.00 | 1.00 | 1.00 | 1.00 | 1.00 | 1.00 | 1 |
| 7 | 1 | 1 | 1 | 1 | 1 | 1 | 1.14 | 1.00 | 1.00 | 1.00 | 1.00 | 1.00 | 1.00 | 1.00 | 1.00 | 1 |
| 8 | 1 | 1 | 1 | 1 | 1 | 1 | 1 | 1.14 | 1.00 | 1.00 | 1.00 | 1.00 | 1.00 | 1.00 | 1.00 | 1.00 |
| 9 | 1 | 1 | 1 | 1 | 1 | 1 | 1 | 1 | 1.14 | 1.00 | 1.00 | 1.00 | 1.00 | 1.00 | 1.00 | 1 |
| 10 | 1 | 1 | 1 | 1 | 1 | 1 | 1 | 1 | 1 | 1.14 | 1.00 | 1.00 | 1.00 | 1.00 | 1.00 | 1 |
| 11 | 1 | 1 | 1 | 1 | 1 | 1 | 1 | 1 | 1 | 1 | 1.14 | 1.00 | 1.00 | 1.00 | 1.00 | 1 |
| 12 | 1 | 1 | 1 | 1 | 1 | 1 | 1 | 1 | 1 | 1 | 1 | 1.14 | 1.00 | 1.00 | 1.00 | 1.00 |
| 13 | 1 | 1 | 1 | 1 | 1 | 1 | 1 | 1 | 1 | 1 | 1 | 1 | 1.14 | 1.00 | 1.00 | 1.00 |
| 14 | 1 | 1 | 1 | 1 | 1 | 1 | 1 | 1 | 1 | 1 | 1 | 1 | 1 | 1.14 | 1.00 | 1.00 |
| 15 | 1 | 1 | 1 | 1 | 1 | 1 | 1 | 1 | 1 | 1 | 1 | 1 | 1 | 1 | 1.14 | 1.00 |
| 16 | 1 | 1 | 1 | 1 | 1 | 1 | 1 | 1 | 1 | 1 | 1 | 1 | 1 | 1 | 1 | 1.14 |
| 17 | 1 | 1 | 1 | 1 | 1 | 1 | 1 | 1 | 1 | 1 | 1 | 1 | 1 | 1 | 1 | 1 |

Women's Health Initiative Study: Combination HRT (estrogen and progesterone)

| | | | | | | | | | | | | | | | | |
|---|---|---|---|---|---|---|---|---|---|---|---|---|---|---|---|---|
| 0 | 1 | 1 | 1 | 1 | 1 | 1 | 1 | 1 | 1 | 1 | 1 | 1 | 1 | 1 | 1 | 1 |
| 1 | 1 | 1.00 | 1.00 | 1.00 | 1.00 | 1.00 | 1.00 | 1.00 | 1.00 | 1.00 | 1.00 | 1.00 | 1.00 | 1.00 | 1.00 | 1.00 |
| 2 | 1 | 1.14 | 1.13 | 1.13 | 1.13 | 1.13 | 1.13 | 1.13 | 1.13 | 1.13 | 1.13 | 1.13 | 1.13 | 1.13 | 1.13 | 1.13 |
| 3 | 1 | 1 | 1.14 | 1.26 | 1.26 | 1.26 | 1.26 | 1.26 | 1.26 | 1.26 | 1.26 | 1.26 | 1.26 | 1.26 | 1.26 | 1.26 |
| 4 | 1 | 1 | 1 | 1.14 | 1.31 | 1.31 | 1.31 | 1.31 | 1.31 | 1.31 | 1.31 | 1.31 | 1.31 | 1.31 | 1.31 | 1.308 |
| 5 | 1 | 1 | 1 | 1 | 1.14 | 1.36 | 1.36 | 1.36 | 1.36 | 1.36 | 1.36 | 1.36 | 1.36 | 1.36 | 1.36 | 1.356 |
| 6 | 1 | 1 | 1 | 1 | 1 | 1.14 | 1.40 | 1.40 | 1.40 | 1.40 | 1.40 | 1.40 | 1.40 | 1.40 | 1.40 | 1.404 |
| 7 | 1 | 1 | 1 | 1 | 1 | 1 | 1.14 | 1.45 | 1.45 | 1.45 | 1.45 | 1.45 | 1.45 | 1.45 | 1.45 | 1.452 |
| 8 | 1 | 1 | 1 | 1 | 1 | 1 | 1 | 1.14 | 1.50 | 1.50 | 1.50 | 1.50 | 1.50 | 1.50 | 1.50 | 1.50 |
| 9 | 1 | 1 | 1 | 1 | 1 | 1 | 1 | 1 | 1.14 | 1.55 | 1.55 | 1.55 | 1.55 | 1.55 | 1.55 | 1.55 |
| 10 | 1 | 1 | 1 | 1 | 1 | 1 | 1 | 1 | 1 | 1.14 | 1.60 | 1.60 | 1.60 | 1.60 | 1.60 | 1.6 |
| 11 | 1 | 1 | 1 | 1 | 1 | 1 | 1 | 1 | 1 | 1 | 1.14 | 1.65 | 1.65 | 1.65 | 1.65 | 1.65 |
| 12 | 1 | 1 | 1 | 1 | 1 | 1 | 1 | 1 | 1 | 1 | 1 | 1.14 | 1.70 | 1.70 | 1.70 | 1.70 |
| 13 | 1 | 1 | 1 | 1 | 1 | 1 | 1 | 1 | 1 | 1 | 1 | 1 | 1.14 | 1.70 | 1.70 | 1.70 |
| 14 | 1 | 1 | 1 | 1 | 1 | 1 | 1 | 1 | 1 | 1 | 1 | 1 | 1 | 1.14 | 1.70 | 1.70 |
| 15 | 1 | 1 | 1 | 1 | 1 | 1 | 1 | 1 | 1 | 1 | 1 | 1 | 1 | 1 | 1.14 | 1.70 |
| 16 | 1 | 1 | 1 | 1 | 1 | 1 | 1 | 1 | 1 | 1 | 1 | 1 | 1 | 1 | 1 | 1.14 |
| 17 | 1 | 1 | 1 | 1 | 1 | 1 | 1 | 1 | 1 | 1 | 1 | 1 | 1 | 1 | 1 | 1 |

Values in bold are published relative risks. Other values are estimated risks derived from published values

APPENDIX F
Estimates of breast cancer relative risk with current HRT use.

| | WHI⁹ | WHI-E¹¹ | | Million Women⁵ | Beral et al⁸ |
|---|---|---|---|---|---|
| Study type | RCT | RCT | | Cohort | Meta-analysis |
| Year of Study | 2002 | 2004 | | 2003 | 1997 |
| Number in | 161,809 | 10,739 | | 1,084,110 | 108,411 |
| Study | | | | | |
| Number on | 8,506 | 5,310 | 139,596 | 115,383 | 17,830 |
| HRT | | | | | |
| HRT used | E + P | E-only | E + P | E-only | Mainly E-only |
| Breast cancer | | | 1.66 | 1.30 | |
| RR. | | | (1.60-1.72) | (1.21-1.40) | |
| (all current | | | | | |
| users) | | | | | |
| <1 year use | | | 1.45 | 0.81 | |
| | | | (1.19-1.78) | (0.55-1.20) | |
| 1-4 years | 1.26 | | 1.74 | 1.25 | |
| | (1.00-1.59) | | (1.60-1.89) | (1.10-1.41) | |
| 5-9 years | | 0.77 | 2.17 | 1.32 | 1.35 |
| | | (0.59-1.01) | (2.03-2.33) | (1.20-1.46) | (1.21-1.49) |
| >=10 years | | | 2.31 | 1.37 | |
| | | | (2.08-2.56) | (1.22-1.54) | |

95% confidence intervals in parenthesis
RCT: Randomized controlled trial
E and P: Oestrogen and progesterone HRT
E-only: Oestrogen-only HRT
RR: Relative risk
WHI: Women's Health Initiative Study

APPENDIX G

| Age | Family History Code | | | | | | | | | |
|---|---|---|---|---|---|---|---|---|---|---|
| (Years) | 1 | 2 | 3 | 4 | 5 | 6 | 7 | 8 | 9 | 10 |
| Under 5 | 1 | 2.5 | 1.6 | 3.3 | 3 | 1.8 | 3.6 | 3.1 | 1.7 | 2.4 |
| 5-9 | 1 | 2.5 | 1.6 | 3.3 | 3 | 1.8 | 3.6 | 3.1 | 1.7 | 2.4 |
| 10-14 | 1 | 2.5 | 1.6 | 3.3 | 3 | 1.8 | 3.6 | 3.1 | 1.7 | 2.4 |
| 15-19 | 1 | 2.5 | 1.6 | 3.3 | 3 | 1.8 | 3.6 | 3.1 | 1.7 | 2.4 |
| 20-24 | 1 | 2.5 | 1.6 | 3.3 | 3 | 1.8 | 3.6 | 3.1 | 1.7 | 2.4 |
| 25-29 | 1 | 2.5 | 1.6 | 3.3 | 3 | 1.8 | 3.6 | 3.1 | 1.7 | 2.4 |
| 30-34 | 1 | 2.5 | 1.6 | 3.3 | 3 | 1.8 | 3.6 | 3.1 | 1.7 | 2.4 |
| 35-39 | 1 | 2.5 | 1.6 | 3.3 | 3 | 1.8 | 3.6 | 3.1 | 1.7 | 2.4 |
| 40-44 | 1 | 2.5 | 1.6 | 3.3 | 3 | 1.8 | 3.6 | 3.1 | 1.7 | 2.4 |
| 45-49 | 1 | 2.5 | 1.6 | 3.3 | 3 | 1.8 | 3.6 | 3.1 | 1.7 | 2.4 |
| 50-54 | 1 | 1.7 | 1.7 | 1.8 | 1.9 | 1.8 | 3.6 | 2.5 | 1.6 | 2.2 |
| 55-59 | 1 | 1.7 | 1.7 | 1.8 | 1.9 | 1.8 | 3.6 | 2.5 | 1.6 | 2.2 |
| 60-64 | 1 | 1.7 | 1.7 | 1.8 | 1.9 | 1.8 | 3.6 | 2.5 | 1.6 | 2.2 |
| 65-69 | 1 | 1.7 | 1.7 | 1.8 | 1.9 | 1.8 | 3.6 | 2.5 | 1.6 | 2.2 |
| 70-74 | 1 | 1.7 | 1.7 | 1.8 | 1.9 | 1.8 | 3.6 | 2.5 | 1.6 | 2.2 |
| 75-79 | 1 | 1.7 | 1.7 | 1.8 | 1.9 | 1.8 | 3.6 | 2.5 | 1.6 | 2.2 |
| 80-84 | 1 | 1.7 | 1.7 | 1.8 | 1.9 | 1.8 | 3.6 | 2.5 | 1.6 | 2.2 |
| 85+ | 1 | 1.7 | 1.7 | 1.8 | 1.9 | 1.8 | 3.6 | 2.5 | 1.6 | 2.2 |

| | |
|---|---|
| FH | |
| code | Family History |
| 1 | No family members with breast cancer |
| 2 | Mother who developed breast cancer age <50 years |
| 3 | Mother who developed breast cancer age 50 years or older |
| 4 | Sister who developed breast cancer age <50 years |
| 5 | Sister who developed breast cancer age 50 years or older |
| 6 | Daughter with breast cancer |
| 7 | Mother and a sister with breast cancer |
| 8 | One first degree relative (mother, sister or daughter) and one |
| | second-degree relative |
| | (aunt or grandmother) with breast cancer |
| 9 | One second degree relative (aunt or grandmother) with |
| | breast cancer |
| 10 | Two second degree relative (aunt or grandmother) with |
| | breast cancer |

APPENDIX H
Cumulative Absolute Risk and Additional Risk of Breast Cancer with HRT Use

| | | | | Additional risk with | | | | Additional risk with | | | |
|---|---|---|---|---|---|---|---|---|---|---|---|
| | | | | combination HRT* | | | | oestrogen-only HRT* | | | |
| Exact | | | | (years of use) | | | | (years of use) | | | |
| Age of | Age | No HRT | | 3 | 5 | 10 | 15 | 3 | 5 | 10 | 15 |
| Woman | Range | Use | | years | Years | years | years | years | years | years | years |
| (years) | (years) | 1 in | % | % | % | % | % | % | % | % | % |
| 40 | 40-79 | 14 | 7.21 | 0.18 | 0.38 | 1.18 | 2.22 | 0.05 | 0.12 | 0.34 | 0.64 |
| 45 | 45-79 | 15 | 6.76 | 0.26 | 0.52 | 1.45 | 2.54 | 0.07 | 0.15 | 0.41 | 0.73 |
| 50 | 50-79 | 16 | 6.10 | 0.31 | 0.60 | 1.59 | 2.82 | 0.09 | 0.18 | 0.45 | 0.81 |
| 55 | 55-79 | 19 | 5.30 | 0.33 | 0.64 | 1.76 | 3.17 | 0.09 | 0.19 | 0.50 | 0.91 |
| 60 | 60-79 | 23 | 4.44 | 0.37 | 0.73 | 2.01 | 3.51 | 0.10 | 0.21 | 0.57 | 1.00 |
| 65 | 65-79 | 29 | 3.48 | 0.42 | 0.84 | 2.19 | 3.27 | 0.12 | 0.25 | 0.62 | 0.91 |
| 70 | 70-79 | 42 | 2.37 | 0.47 | 0.88 | 1.84 | — | 0.13 | 0.25 | 0.50 | — |
| 75 | 75-79 | 88 | 1.14 | 0.43 | 0.58 | — | — | 0.12 | 0.14 | — | — |

1. A method of determining cumulative absolute breast cancer risk for a patient, said method comprising the steps of:
determining underlying population breast cancer incidence associated with said patient;
determining baseline age specific breast cancer incidence for said patient based on said underlying population breast cancer incidence; and
determining the cumulative absolute breast cancer risk for said patient based on the baseline age specific breast cancer incidence determined for said patient, wherein the underlying population breast cancer incidence associated with said patient is adjusted for hormone replacement therapy (HRT) use.

2. The method according to claim 1, further including the step of removing the effect of HRT use from population breast cancer incidence data prior to determining the underlying population breast cancer incidence associated with said patient.

3. The method according to claim 2, wherein the effect of HRT use is determined based on direct or indirect methods.

4. The method according to claim 1, further including the step of adjusting the underlying population breast cancer incidence associated with said patient based on family history of said patient.

5. The method according to claim 4, wherein the effect of family history of said patient on the underlying population breast cancer incidence may be determined based on direct and indirect methods.

6. The method according to claim 1, further including the step of adjusting the underlying population breast cancer incidence associated with said patient based on age of said patient.

7. The method according to claim 1, wherein the cumulative absolute breast cancer risk for said patient is determined based on length of time of HRT use.

8. The method according to claim 1, wherein the cumulative absolute breast cancer risk for said patient is determined based on type of HRT.

9. The method according to claim 1, further comprising the step of adjusting age specific breast cancer incidence for said patient based on breast cancer screening.

10. The method according to claim 1, further comprising the step of selecting HRT risk study data for use in determining the cumulative absolute breast cancer risk for said patient.

11. The method according to claim 1, further comprising the step of providing risk data for use in determining the cumulative absolute breast cancer risk for said patient.

12-29. (canceled)
